(19) United States
(12) Patent Application Publication
(10) Pub. No.: US 2015/0024502 A1
(43) Pub. Date: Jan. 22, 2015

(54) METHODS OF STABILIZING TOTAL ORGANIC CARBON (TOC) LEVELS IN NON-ACIDIFIED STANDARDS FOR TOC ANALYZERS AT ROOM TEMPERATURE

(71) Applicant: GENERAL ELECTRIC COMPANY, Schenectady, NY (US)

(72) Inventor: Shouquan Deng, ShangHai (CN)

(21) Appl. No.: 14/371,290

(22) PCT Filed: Jan. 9, 2012
(86) PCT No.: PCT/CN2012/070130

§ 371 Date: Aug. 5, 2014

(51) Int. Cl. G01N 33/18

(52) U.S. Cl. CPC G01N 33/1846
USPC 436/18

(57) ABSTRACT

Methods for stabilizing reference standards used in total organic carbon (TOC) analysis of water and pharmaceutical samples are disclosed. The methods include adding a preservative to TOC reference standards. The preservative is selected from the group consisting of copper (II) sulfate and hydrogen sulfite. Compositions of the stabilized reference standards are also disclosed.

### FIELD OF THE INVENTION

The field of the invention generally relates to standards used in total organic carbon analysis of water and pharmaceutical processes. The invention also pertains to chemical compositions for use in total organic carbon analysis.

### BACKGROUND OF THE INVENTION

Water quality and cleanliness of food and pharmaceutical grade processing equipment is often indicated by the total organic carbon (TOC) present in the sample. Briefly, TOC analyzers measure TOC by converting organic carbon in the sample to CO₂ through oxidation or acidification. The CO₂ is then measured.

United States Pharmacopeia (USP) requires TOC analyzers to be able to distinguish between organic and inorganic carbon. USP also requires TOC analyzer users to calibrate their equipment and demonstrate its suitability for TOC analysis. USP has selected sucrose and benzoquinone as the reference standards to demonstrate TOC analyzer suitability. USP Chapters 11 and 643 describe the reference standards and how to demonstrate TOC analyzer suitability.

USP Chapter 643 has special definitions for the reference standards used to demonstrate TOC analyzer suitability. This specification defines the reference standards similarly. The sucrose reference standard is defined as a “standard solution” with a concentration of about 1.2 mg of sucrose per liter of water. The benzoquinone solution is defined as a “system suitability solution” with a concentration of about 0.75 mg of 1,4-benzoquinone per liter of water. “Reagent water” is defined as having a TOC level of not more than 0.10 mg per liter is used a control.

According to USP Chapter 643, the first step in demonstrating TOC analyzer suitability is to test the reagent water in the analyzer and record the response, r_{w}. The test is repeated with the standard solution and the response, rₛ is recorded. The next step is to determine the theoretical response of the analyzer. The theoretical response is equal to the response of the standard solution minus the response to the reagent water (rₛ−r_{w}). Next, the system suitability solution is tested with the analyzer and the response, rₛₛ, is recorded.

Finally, the response efficiency of the system suitability solution is calculated using equation I:

100[(rₛₛ−r_{w})/(rₛ−r_{w})] (I)

A TOC analyzer is suitable if the response efficiency of the system suitability solution not less than 85% and not more than 115% of the theoretical response.

The TOC of sucrose and benzoquinone standards, however, deteriorates with time, temperature, and exposure to light. TOC deterioration in the standards can cause an otherwise functioning TOC analyzer to fail calibration or suitability tests. To minimize the changes in TOC of these standards, many standard manufacturers ship the standards to customers overnight in vacuumed Aluminum bags and recommend refrigeration upon receipt. These standards also have an expiration date. Unfortunately, the standards will still experience temperature fluctuations during shipping. The temperature fluctuations may be extreme. Moreover, the shipping time may vary depending on the location of the customer. For example, the TOC levels in standards deteriorate when shipped from the U.S. to China. These standards are kept at ambient conditions while waiting to clear customs in China, leading to TOC deterioration. Thus there is a need for standards that maintain their TOC levels over time, even under extreme shipping conditions.

### BRIEF DESCRIPTION OF THE INVENTION

It was surprisingly discovered that inorganic preservatives may be added to total organic carbon (TOC) reference standards to improve TOC stability of the standards without affecting the standard's response during calibration and suitability tests. Suitable preservatives may be any inorganic material that stabilizes the standards through killing bacteria or scavenging oxidants present in the standards.

In one embodiment, a method of stabilizing a reference standard used in total organic carbon (TOC) analysis is disclosed. The method comprises providing a solution of a reference standard and adding a preservative to the solution to create a stabilized reference standard.

In another embodiment, the solution is a standard solution with a sucrose concentration of about 1.2 mg of sucrose per liter of water. In yet another embodiment, the solution is a system suitability solution with a 1,4-benzoquinone concentration of about 0.75 mg of 1,4-benzoquinone per liter of water.

Another embodiment further discloses a preservative selected from the group consisting of copper (II) sulfate and hydrogen sulfite. In another embodiment, the preservative is copper (II) sulfate wherein the concentration of copper (II) sulfate in the solution ranges from about 0.05 to about 0.20 mg Cu²⁺ per liter of said solution. Alternatively, the concentration of copper (II) sulfate ranges from about 0.08 to about 0.12 mg Cu²⁺ per liter of said solution. In another embodiment, the preservative is hydrogen sulfite wherein the concentration of hydrogen sulfite in the solution ranges from about 0.05 to 0.20 mg HSO₃⁻ per liter of said solution. Alternatively, the concentration of hydrogen sulfite may range from about 0.08 to about 0.12 mg HSO₃⁻ per liter of said solution.

In another embodiment, a method for demonstrating TOC analyzer suitability is disclosed using stabilized reference standards.

In another embodiment, a stabilized reference standard for use in total organic carbon analysis is disclosed. The stabilized reference standard comprises a reference standard solution and a preservative. In another embodiment, the reference standard is a standard solution with a sucrose concentration of about 1.2 mg of sucrose per liter of water. In yet another embodiment, the reference standard is a system suitability solution with a 1,4-benzoquinone concentration of about 0.75 mg of 1,4-benzoquinone per liter of water. In another embodiment, the preservative is selected from the group consisting of copper (II) sulfate and hydrogen sulfite. In yet another embodiment, the preservative is copper (II) sulfate present in a concentration ranging from about 0.05 to about 0.20 mg Cu²⁺ per liter of the reference standard solution. Alternatively, the concentration of the copper (II) sulfate preservative ranges from about 0.08 to 0.12 mg Cu²⁺ per liter of the reference standard solution. In yet another embodiment, the preservative is hydrogen sulfite present in a concentration ranging from about 0.05 to about 0.20 mg HSO₃⁻ per liter of the reference standard solution. Alternatively, the concentration of the hydrogen sulfite preservative is present in a concentration ranging from about 0.08 to 0.12 mg HSO₃⁻ per liter of reference standard solution.

### BRIEF DESCRIPTION OF THE DRAWINGS

FIG. 1 is a graph showing the total organic carbon (TOC) responses of various TOC reference standards with time.

### DETAILED DESCRIPTION OF EXEMPLARY EMBODIMENTS

It was surprising discovered that inorganic preservatives may be added to total organic carbon (TOC) reference standards to improve TOC stability of the standards without affecting the standard's response during calibration and suitability tests. Suitable preservatives may be any inorganic material that stabilizes the standards through killing bacteria or scavenging oxidants present in the standards.

In one embodiment, a method of stabilizing a reference standard used in total organic carbon (TOC) analysis is disclosed. The method comprises providing a solution of a reference standard and adding a preservative to the solution to create a stabilized reference standard.

In another embodiment, the solution is a standard solution with a sucrose concentration of about 1.2 mg of sucrose per liter of water. In yet another embodiment, the solution is a system suitability solution with a 1,4-benzoquinone concentration of about 0.75 mg of 1,4-benzoquinone per liter of water.

Another embodiment further discloses a preservative selected from the group consisting of copper (II) sulfate and hydrogen sulfite. In another embodiment, the concentration of copper (II) sulfate in the solution ranges from about 0.05 to about 0.20 mg Cu²⁺ per liter of said solution. Alternatively, the concentration of copper (II) sulfate ranges from about 0.08 to about 0.12 mg Cu²⁺ per liter of said solution. In another embodiment, the concentration of hydrogen sulfite in the solution ranges from about 0.05 to 0.20 mg HSO₃⁻ per liter of said solution. Alternatively, the concentration of hydrogen sulfite may range from about 0.08 to about 0.12 mg HSO₃⁻ per liter of said solution.

In another embodiment, a method for demonstrating TOC analyzer suitability is disclosed using stabilized reference standards.

In another embodiment, a stabilized reference standard for use in total organic carbon analysis is disclosed. The stabilized reference standard comprises a reference standard solution and a preservative. In another embodiment, the reference standard is a standard solution with a sucrose concentration of about 1.2 mg of sucrose per liter of water. In yet another embodiment, the reference standard is a system suitability solution with a 1,4-benzoquinone concentration of about 0.75 mg of 1,4-benzoquinone per liter of water. In another embodiment, the preservative is selected from the group consisting of copper (II) sulfate and hydrogen sulfite. In yet another embodiment, the preservative is copper (II) sulfate present in a concentration ranging from about 0.05 to about 0.20 mg Cu²⁺ per liter of the reference standard solution. Alternatively, the concentration of the copper (II) sulfate preservative ranges from about 0.08 to 0.12 mg Cu²⁺ per liter of the reference standard solution. In yet another embodiment, the preservative is hydrogen sulfite present in a concentration ranging from about 0.05 to about 0.20 mg HSO₃⁻ per liter of the reference standard solution. Alternatively, the concentration of the hydrogen sulfite preservative is present in a concentration ranging from about 0.08 to 0.12 mg HSO₃⁻ per liter of reference standard solution.

### EXAMPLES

### Comparative Example 1

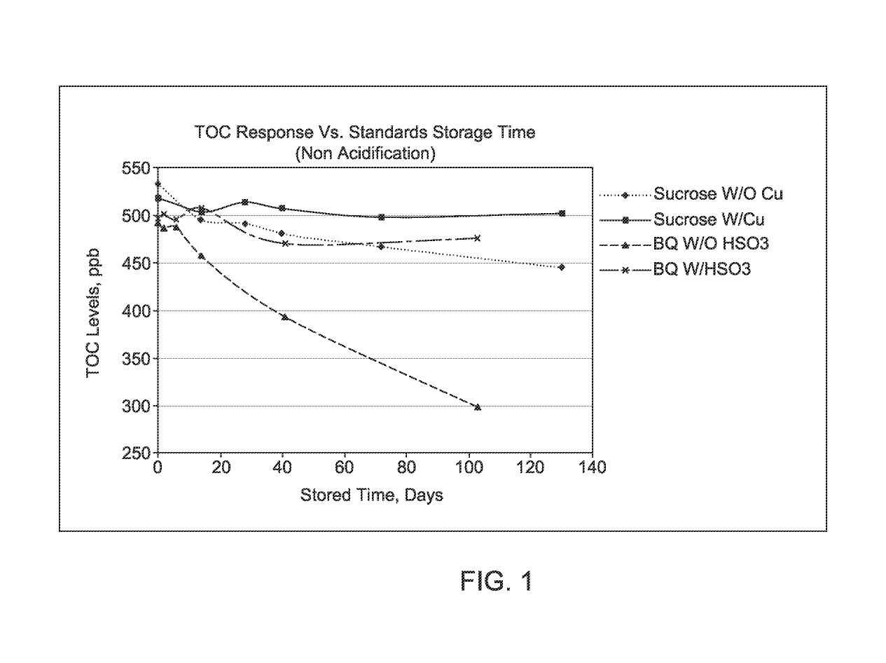

### FIG. 1, Sucrose W/O Cu

A non-acidified sucrose standard solution without copper (II) sulfate was added to a 500 mL-flask and retained as a control. The flask was stored at room conditions (25° C. and exposure to light). The TOC response of the control standard solution was measured over time.

### Example 1

### FIG. 1, Sucrose W/Cu

Low levels of copper (II) sulfate, or CuSO₄ (about 0.10 mg Cu²⁺/L) were added to a 500 mL-flask with a non-acidified sucrose standard. The flask was stored at room conditions (25° C. and exposure to light). The TOC response of the solution was measured over time. The TOC levels remained stabilized for 4 months.

### Comparative Example 2

### FIG. 1, BQ W/O HSO3

A non-acidified benzoquinone standard without hydrogen sulfite was added to a 500 mL-flask and retained as a control. The flask was stored at room conditions (25° C. and exposure to light). The TOC response of the control standard solution was measured over time.

### Example 2

### FIG. 1, BQ W/HSO3

Low levels of hydrogen sulfite, or HSO₃ (about 0.10 mg HSO₃⁻/L) were added to a 500 mL-flask with a non-acidified benzoquinone standard. The flask was stored at room conditions (25° C. and exposure to light). The TOC response of the solution was measured over time. The TOC levels remained stabilized for 3 months.

The TOC responses over time for the above samples are shown in FIG. 1.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method of stabilizing a reference standard used in total organic carbon (TOC) analysis comprising:
(a) providing a solution of said reference standard; and
(b) adding a preservative to said solution to create a stabilized reference standard.

2. The method of claim 1, wherein said solution is a standard solution with a sucrose concentration of about 1.2 mg of sucrose per liter of water.

3. The method of claim 1, wherein said solution is a system suitability solution with a 1,4-benzoquinone concentration of about 0.75 mg of 1,4-benzoquinone per liter of water.

4. The method of claim 1, wherein said preservative is selected from the group consisting of copper (II) sulfate and hydrogen sulfite.

5. The method of claim 4, wherein said preservative is copper (II) sulfate present in a concentration in said solution in a range from about 0.05 to about 0.20 mg Cu²⁺ per liter of said solution.

6. The method of claim 4, wherein said preservative is copper (II) sulfate present in a concentration in said solution in a range from about 0.08 to about 0.12 mg Cu²⁺ per liter of said solution.

7. The method of claim 4, wherein said preservative is hydrogen sulfite present in a concentration in said solution in a range from about 0.05 to about 0.20 mg HSO₃⁻ per liter of said solution.

8. The method of claim 4, wherein said preservative is hydrogen sulfite present in a concentration in said solution in a range from about 0.08 to about 0.12 mg HSO₃⁻ per liter of said solution.

9. A stabilized reference standard for use in total organic carbon analysis comprising:
(c) a reference standard solution; and
(d) a preservative.

10. The stabilized reference standard of claim 9, wherein said reference standard solution is a standard solution with a sucrose concentration of about 1.2 mg of sucrose per liter of water.

11. The stabilized reference standard of claim 9, wherein said reference standard solution is a system suitability solution with a 1,4-benzoquinone concentration of about 0.75 mg of 1,4-benzoquinone per liter of water.

12. The stabilized reference standard of claim 9, wherein said preservative is selected from the group consisting of copper (II) sulfate and hydrogen sulfite.

13. The stabilized reference standard of claim 12, wherein said preservative is copper (II) sulfate present in a concentration in said solution in a range from about 0.05 to about 0.20 mg Cu²⁺ per liter of said solution.

14. The stabilized reference standard of claim 12, wherein said preservative is copper (II) sulfate present in a concentration in said solution in a range from about 0.08 to about 0.12 mg Cu²⁺ per liter of said solution.

15. The stabilized reference standard of claim 12, wherein said preservative is hydrogen sulfite present in a concentration in said solution in a range from about 0.05 to about 0.20 mg HSO₃⁻ per liter of said solution.

16. The stabilized reference standard of claim 12, wherein said preservative is hydrogen sulfite present in a concentration in said solution in a range from about 0.08 to about 0.12 mg HSO₃⁻ per liter of said solution.
